(19) United States
(12) Patent Application Publication
(10) Pub. No.: US 2018/0116556 A1
(43) Pub. Date: May 3, 2018

(54) HEIGHT MEASUREMENT METHOD BASED ON MONOCULAR MACHINE VISION

(71) Applicant: Nanjing AvatarMind Robot Technology Co., Ltd., Nanjing (CN)

(72) Inventors: Fan ZHANG, Nanjing (CN); Haizhou WU, Nanjing (CN)

(21) Appl. No.: 15/806,308

(22) Filed: Nov. 7, 2017

Related U.S. Application Data
(63) Continuation of application No. PCT/CN2017/102997, filed on Sep. 22, 2017.

(30) Foreign Application Priority Data
Oct. 27, 2016 (CN) 201610955233.6

(51) Int. Cl. A61B 5/107; G01B 11/03; G06K 9/00; A61B 5/00; G01B 11/28; A41H 1/02

(52) U.S. Cl. CPC A61B 5/1072; G01B 11/03; A41H 1/02; A61B 5/0064; G01B 11/28; G06K 9/00234

(57) ABSTRACT

The present disclosure provides a height measurement method based on monocular machine vision. The method includes: picking up, by an RGB camera arranged on the head of a robot, a two-dimensional identifier from the head to feet of a person under measurement; calculating, by the robot, a homography matrix of a current visual field according to four corner points on the visual location identifier; acquiring a head image region by segmenting the image, and calculating pixel coordinates of a head vertex; and calculating a height of the person under measurement. The height measurement method based on monocular machine vision according to the present disclosure is simple in operation and calculation. The height of a person under measurement may be measured by himself or herself with no assistance from others. The measurement method features non-contact. The method further improves the measurement precision, and enhances the measurement speed.

This application is an US national stage application of the international patent application PCT/CN2017/102997, filed on Sep. 22, 2017, which is based upon and claims priority of Chinese Patent Application No. 201610955233.6, filed before Chinese Patent Office on Oct. 27, 2016 and entitled “HEIGHT MEASUREMENT METHOD BASED ON MONOCULAR MACHINE VISION”, the entire contents of which are incorporated herein by reference.

### TECHNICAL FIELD

The present disclosure relates to the technical field of length measurement, and in particular, relates to a height measurement method based on monocular machine vision.

### BACKGROUND

Considering various needs such as physical examination, selection of players and the like, heights of persons need to be measured. The current height measurement method generally uses rulers and benchmarks. In such measurement method, the operations are not convenient, direct contacts to the bodies are needed, automatic measurement may not be implemented, and errors may be simply caused due to improper operations.

### SUMMARY

The technical problem to be solved by the present disclosure is to provide a height measurement method based on monocular machine vision. This method is capable of implementing automatic non-contact measurement of the heights of human bodies, and features convenient operations and precise measurement.

To achieve the above objective, the present disclosure provides a height measurement method based on monocular machine vision. The method includes the following steps:

causing a person under measurement to stand in a specified region on a planar identifier;

maintaining the head of a robot in a horizontal state, and adjusting a distance between the robot and the person under measurement such that an RGB camera arranged on the head of the robot picks up a two-dimensional identifier from the head to the feet of the person under measurement;

calculating, by the robot, a homography matrix H=M[r1,r2,r3,t] of a current visual field according to four corner points on the two-dimensional identifier based on the following predefined equations:

$$[ \begin{matrix}x \\ y \\ 1\end{matrix} ] = sM [ r 1 , r 2 , r 3 , t ] [ \begin{matrix}X \\ Y \\ Z \\ 1\end{matrix} ] = sM [ r 1 , r 2 , t ] [ \begin{matrix}X \\ Y \\ 1\end{matrix} ] ,$$

wherein (x, y, 1) denotes homogenous coordinates of any corner point on the visual location identifier in pixel coordinates in an image coordinate system of the camera; (X, Y, Z, 1) denotes homogenous coordinates of the corner point in the coordinate system of the visual location identifier; assume that a plane of the visual location identifier Z is equal to 0, then the homogenous coordinates of the corner point in the visual location identifier coordinate system are simplified as (X, Y, 0, 1); s denotes any introduced scale proportion parameter; M denotes an internal parameter matrix of the camera; r1, r2 and r3 denote three column vectors in a rotary matrix a visual location identifier coordinate system relative to the image coordinate system of the camera; and t denotes a translation vector;

acquiring a head image region via segmentation based on an image segmentation algorithm, and calculating pixel coordinates (x0, y0) of a head vertex of the person under measurement; and

according to the homography matrix, calculating a height Z of the person under measurement by substituting x=x0, y=y0 and X=0 into the following predefined equations:

$$[ \begin{matrix}x \\ y \\ 1\end{matrix} ] = sM [ r 1 , r 2 , r 3 , t ] [ \begin{matrix}X \\ Y \\ Z \\ 1\end{matrix} ] .$$

The pixel coordinates of the head vertex of the person under measurement may be calculated by the following three steps:

(1) detecting a face rectangular region in the image using the Haar-Adaboost face detection algorithm; and identifying the face rectangular region in the image by a face image sample trained face detector based on the Haar-Adaboost face detection algorithm;

(2) acquiring the head region via segmentation based on the Watershed algorithm; wherein the face rectangular region may be marked as a foreground image region after the face rectangular region is identified, non-face background regions on both sides of the face may be marked as background image regions, and a head profile of the person under measurement may be completely acquired via segmentation from the background based on the watershed segmentation algorithm; and

(3) obtaining the pixel coordinates of the head of the person under measurement; wherein it is defaulted that the head of the person under measurement maintains upright, the pixel coordinate x0 in the x-axis direction of the head vertex is equal to the x-axis coordinate value at the center of the face rectangular region; the watershed segmentation algorithm is capable of segmenting an integral profile of the head, the pixel coordinate y0 of the head vertex in the y-axis direction may be obtained by calculating an average value of the y-axis coordinate values of the head profile point of the x-axis coordinate within the range of (x0−Δx, x0+Δx).

The height measurement method based on monocular machine vision according to the present disclosure is simple in operation and calculation. The height of a person under measurement may be measured by himself or herself with no assistance from others. The measurement method features non-contact. The method further improves the measurement precision, and enhances the measurement speed.

### BRIEF DESCRIPTION OF THE DRAWINGS

FIG. 1 is a schematic structural diagram of a robot used in a height measurement method according to the present disclosure;

FIG. 2 is a schematic diagram of a measurement region during use of the height measurement method according to the present disclosure; and

FIG. 3 is a schematic diagram of extracting a head profile image according to an embodiment of the present disclosure.

### DETAILED DESCRIPTION

Hereinafter a height measurement method based on monocular machine vision according to the present disclosure is described in detail with reference to the accompanying drawings.

The present disclosure provides a height measurement method based on monocular machine vision. The method includes the following steps:

causing a person under measurement to stand in a specified region on a planar identifier;

maintaining the head of a robot in a horizontal state, and adjusting a distance between the robot and the person under measurement such that an RGB camera arranged on the head of the robot picks up a two-dimensional identifier from the head to the feet of the person under measurement;

calculating, by the robot, a homography matrix H=SM[1,r2,r3,t] of a current visual field according to four corner points on the two-dimensional identifier based on the following predefined equations:

$$[ \begin{matrix}x \\ y \\ 1\end{matrix} ] = sM [ r 1 , r 2 , r 3 , t ] [ \begin{matrix}X \\ Y \\ Z \\ 1\end{matrix} ] = sM [ r 1 , r 2 , t ] [ \begin{matrix}X \\ Y \\ 1\end{matrix} ] ,$$

wherein (x, y, 1) denotes homogenous coordinates of any corner point on the visual location identifier (that is, the planar identifier) in pixel coordinates in an image coordinate system of the camera; (X, Y, Z, 1) denotes homogenous coordinates (positions of the four corner points are known, and therefore the homogenous coordinates thereof in the visual location identifier coordinate system are predefined) of the corner point in the coordinate system of the visual location identifier; s denotes any introduced scale proportion parameter; M denotes an internal parameter matrix of the camera; r1, r2 and r3 denote three column vectors in a rotary matrix a visual location identifier coordinate system relative to the image coordinate system of the camera; and t denotes a translation vector;

assume that a plane of the visual location identifier Z is equal to 0, then the homogenous coordinates of the corner point in the visual location identifier coordinate system are simplified as (X, Y, 0, 1), and the homography matrix is transformable, such that r1, r2 and t may be calculated according to the four corner points; r1, r2 and r3 are all column vectors of a rotary matrix R, and since the rotary matrix R is a unitary orthogonal matrix, r1, r2 and r3 are all unitary vectors that are orthogonal to each other; the unitary vector r3 may be calculated based on a cross product of r1 and r2, that is, r3=r1×r2; and

acquiring a head image region via segmentation, and calculating pixel coordinates (x0, y0) of a head vertex of the person under measurement.

The pixel coordinates of the head vertex of the person under measurement may be calculated by the following three steps:

(4) detecting a face rectangular region in the image using the Haar-Adaboost face detection algorithm; and identifying the face rectangular region in the image by a face image sample trained face detector based on the Haar-Adaboost face detection algorithm; and

(5) acquiring the head region via segmentation based on the Watershed algorithm; wherein the face rectangular region may be marked as a foreground image region after the face rectangular region is identified, non-face background regions on both sides of the face may be marked as background image regions, and a head profile of the person under measurement may be completely acquired via segmentation from the background based on the watershed segmentation algorithm;

watershed algorithm refers to the watershed image segmentation algorithm:

this algorithm is capable of automatically acquiring a border profile of two regions via segmentation by means of respectively marking a foreground image region and a background image region; according to the present disclosure, a profile region of the head vertex is acquired via segmentation based on the watershed algorithm, and then the pixel coordinates of an uppermost point of the head vertex are acquired according to the profile of the head vertex; the specific process includes:

a. detecting a face rectangular region 1 using a face detection algorithm;

b. marking the face rectangular region 1 as a foreground image region for segmentation; and

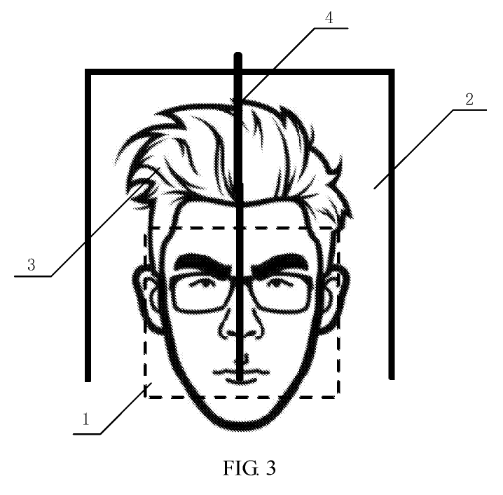

c. marking a background image region not including the head of the person according to size and position of the face rectangular region 1; specifically, position and size of the head profile are concluded according to the face rectangular region, and then the region outside the head profile region is marked as a background image region 3, such that a head image region 3 of the person under measurement is automatically generated, that is, the head profile image (this is a function that can be implemented by the watershed algorithm, which is not described herein any further), as illustrated in FIG. 3; to extract the head profile image, other methods, such as, background matting and the like, may also be used; and

(6) obtaining the pixel coordinates of the head vertex of the person under measurement.

Assume that it has been detected that the pixel coordinates at the center of the face rectangular region are (x1, x2) and the head of the person under measurement maintains upright, then an intersection of a vertical line passing through the center of the face rectangular region and the head vertex profile in the head profile image is a head vertex 4. The pixel coordinates (x0, y0) of the head vertex are found at the head profile, wherein x0=x1.

A height Z of the person under measurement is calculated by substituting x=x0, y=y0, X=0 and Y=Y0 into the following predefined equations:

$$[ \begin{matrix}x \\ y \\ 1\end{matrix} ] = sM [ r 1 , r 2 , r 3 , t ] [ \begin{matrix}X \\ Y \\ Z \\ 1\end{matrix} ] .$$

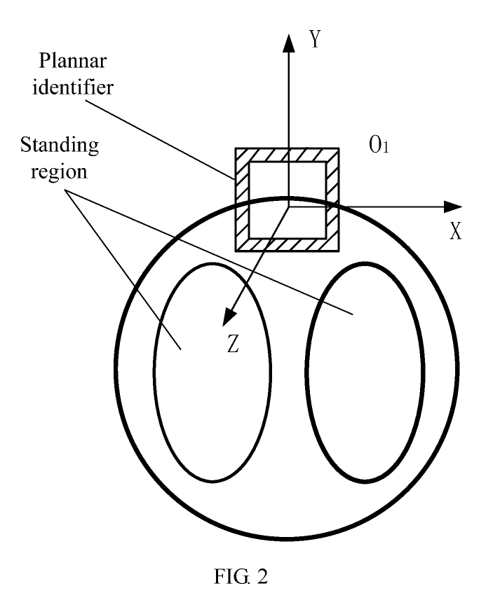

As illustrated in FIG. 2, the position where the person under measurement is fixed relative to a visual location identifier coordinate system O1. Therefore, the coordinates of the central point of the position where the person under measurement stands are also fixed. Assume that the Y-axis coordinate value of the central point in the visual location identifier coordinate system O1 is a constant, Y=Y0, then the X-axis coordinate value is X=0. The person vertically stands at the center of the region, that is, the Y-axis coordinate value of the head vertex of the person in the visual location identifier coordinate system O1 is a constant Y0, then the X-axis coordinate value in the visual location identifier coordinate system O1 is 0. According to the above equations, when the pixel coordinates (x0, y0) of the head vertex are known, and both the X-axis coordinate and the Y-axis coordinate of the head vertex in the planar identifier coordinate system O1 are known, Z (the height of the person under measurement) of the coordinates of the head vertex may be calculated.

As illustrated in FIG. 2, as regards the planar identifier, during height measurement, it should be ensured that the robot sees the planar identifier; as regards the standing region, the person under measurement should stand in this region; and O1 is an identifier coordinate system with the center of the identifier as the origin.

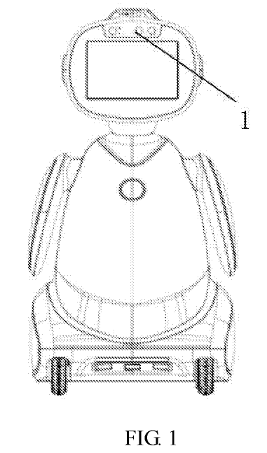

As illustrated in FIG. 1, in the height measurement method based on monocular machine vision according to the present disclosure, a robot is employed, and a RGB camera 1 is arranged on the head of the robot.

The above embodiments are merely used to illustrate the technical solutions of the present disclosure, instead of limiting the protection scope of the present disclosure. Any modification, equivalent replacement, or improvement made without departing from the spirit and principle of the present disclosure should fall within the protection scope defined by the appended claims of the present disclosure.

What is claimed is:

1. A height measurement method based on monocular machine vision, comprising the following steps:
obtaining, by a camera on a robot, an image when a person under measurement stands in a specified region corresponding to a visual location identifier, the image comprising the visual location identifier from the head to the feet of the person under measurement;
calculating, by the robot, a homography matrix of a current visual field according to four corner points on the visual location identifier;
acquiring a head image region by segmenting the image, and calculating pixel coordinates of a head vertex of the person under measurement; and
calculating a height of the person under measurement according to the pixel coordinates of the head vertex of the person under measurement and the homography matrix of the current visual field.

2. The height measurement method based on monocular machine vision according to claim 1, wherein the calculating, by the robot, a homography matrix of a current visual field according to four corner points on the visual location identifier comprises:
substituting each of the corner points into the following predefined equations:
$$[ \begin{matrix}x \\ y \\ 1\end{matrix} ] = sM [ r 1 , r 2 , r 3 , t ] [ \begin{matrix}X \\ Y \\ Z \\ 1\end{matrix} ] ,$$
wherein (x, y, 1) denotes homogenous coordinates of any corner point on the visual location identifier in pixel coordinates in an image coordinate system of the camera; s denotes any introduced scale proportion parameter; M denotes an internal parameter matrix of the camera; r1, r2 and r3 denote three column vectors in a rotary matrix a visual location identifier coordinate system relative to the image coordinate system of the camera; and t denotes a translation vector;
(X, Y, Z, 1) denotes homogenous coordinates of the corner point in the coordinate system of the visual location identifier;
assume that a plane of the visual location identifier Z is equal to 0, then the homogenous coordinates of the corner point in the visual location identifier coordinate system are simplified as (X, Y, 0, 1), and the homography matrix is transformed into:
$$[ \begin{matrix}x \\ y \\ 1\end{matrix} ] = sM [ r 1 , r 2 , r 3 , t ] [ \begin{matrix}X \\ Y \\ Z \\ 1\end{matrix} ] = sM [ r 1 , r 2 , t ] [ \begin{matrix}X \\ Y \\ 1\end{matrix} ] ;$$
the homography matrix of the current visual field is calculated as H=M[r1, r2, r3, t].

3. The height measurement method based on monocular machine vision according to claim 1, wherein the acquiring a head image region by segmenting the image, and calculating pixel coordinates of a head vertex of the person under measurement comprises:
detecting a face rectangular region in the picked-up image using the Haar-Adaboost face detection algorithm;
acquiring the head image region via segmentation based on the Watershed algorithm; and
obtaining the pixel coordinates of the head vertex of the person under measurement according to the rectangular region and the acquired head image region.

4. The height measurement method based on monocular machine vision according to claim 3, wherein the detecting a face rectangular region in the picked-up image using the Haar-Adaboost face detection algorithm comprises:
identifying the face rectangular region in the image by a face image sample trained face detector based on the Haar-Adaboost face detection algorithm.

5. The height measurement method based on monocular machine vision according to claim 3, wherein the acquiring the head image region via segmentation based on the Watershed algorithm comprises:
marking the face rectangular region as a foreground image region after the face rectangular region is identified; and
marking a background image region not including the head of the person according to size and position of the face rectangular region, and obtaining the head image region of the person under measurement.

6. The height measurement method based on monocular machine vision according to claim 3, wherein the obtaining the pixel coordinates of the head vertex of the person under measurement according to the rectangular region and the acquired head image region comprises:
determining an intersection of a central point of the face rectangular region, a vertical line parallel to the y-axis and a head vertex profile in the head image region as the head vertex; and
determining a pixel coordinate in the x-axis direction of the heat vertex of the person under measurement as an x-axis coordinate value of the central point of the face rectangular region.

7. The height measurement method based on monocular machine vision according to claim 1, wherein the calculating a height of the person under measurement according to the pixel coordinates of the head vertex of the person under measurement and the homography matrix of the current visual field comprises:
substituting the pixel coordinates of the head vertex and the homography matrix of the current visual field into the following predefined equations, and calculating the height of the person under measurement:
$$[ \begin{matrix}x \\ y \\ 1\end{matrix} ] = sM [ r 1 , r 2 , r 3 , t ] [ \begin{matrix}X \\ Y \\ Z \\ 1\end{matrix} ] ,$$
wherein x denotes a calculated pixel coordinate of the head vertex in the x-axis direction, y denotes a calculated pixel coordinate of the head vertex in the y-axis direction, X is 0, Y denotes a Y-axis coordinate of the central point in the specified region where the person under measurement in the visual location identifier coordinate system, and Z denotes a height of the person under measurement.
